(19) United States
(12) Patent Application Publication
(10) Pub. No.: US 2025/0262778 A1
(43) Pub. Date: Aug. 21, 2025

(54) STRUCTURAL WAVE FRAMEWORKS WITH NEUTRAL AXIS AND BEND LIMITERS

(71) Applicant: Howard Graham, Temecula, CA (US)

(72) Inventor: Howard Graham, Temecula, CA (US)

(21) Appl. No.: 19/053,883

(22) Filed: Feb. 14, 2025

Related U.S. Application Data
(60) Provisional application No. 63/553,864, filed on Feb. 15, 2024.

(51) Int. Cl. B25J 18/06; A61M 25/01

(52) U.S. Cl. CPC B25J 18/06; A61M 25/0138; A61M 25/0147

(57) ABSTRACT

Flexible Frameworks with Neutral Axis and Bend limiters may be provided by a framework, including a plurality of flexible components, each flexible component having a plurality of structural components, including a first annular member; a second annular member; a first axial member, a second axial member, diametrically opposite from where the first axial member is connected to the first annular member and the second annular member; a first undulating member, having an annular profile and a waveform defined about a circumference thereof, which intersects the first annular member twice, intersects the first axial member once, and intersects the second axial member once; and a second undulating member, having an annular profile and a waveform defined about a circumference thereof, which intersects the second annular member twice, intersects the first axial member once, and intersects the second axial member once.

### CROSS-REFERENCES TO RELATED APPLICATIONS

The present disclosure claims benefit and priority to U.S. Provisional Patent Application 63/553,864, entitled “STRUCTURAL WAVE FRAMEWORKS WITH NEUTRAL AXIS AND DEFLECTION CONSTRAINTS” and filed on 2024 Feb. 15, which is incorporated herein by reference in its entirety.

### BACKGROUND

When an element in bending begins to deflect, portions of the element experience compressive strain, and other portions experience tensile strain. The neutral axis is the axis, or plane, in the cross section of the element in bending where there are no compressive or tensile strains. In uniform elements, the neutral axis typically bisects the element perpendicularly to the direction of the applied bending force.

### SUMMARY

The present disclosure provides structural wave frameworks with neutral axis and bend limiters.

A provided flexible framework includes a plurality of flexible components, each flexible component of the plurality of flexible components having a plurality of structural components, including a first annular member; a second annular member; a first axial member, connected on opposing ends to the first annular member and the second annular member, respectively; and a second axial member, connected on opposing ends to the first annular member and the second annular member, respectively, in some embodiments diametrically opposite from where the first axial member is connected to the first annular member and the second annular member. The flexible framework further includes a first undulating member, having an annular profile and a waveform defined about a circumference thereof, which intersects the first annular member twice, intersects the first axial member once, and intersects the second axial member once; and a second undulating member, having an annular profile and a waveform defined about a circumference thereof, which intersects the second annular member twice, intersects the first axial member once, and intersects the second axial member once.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 is prior art, demonstrating mechanical concepts relevant to the contents of the present disclosure.

FIG. 2 is prior art, demonstrating mechanical concepts relevant to the contents of the present disclosure.

FIG. 3 illustrates a flexible framework component, according to embodiments of the present disclosure.

FIG. 4 illustrates a flexible framework component with dependent bend limiters, according to embodiments of the present disclosure.

FIG. 5 illustrates a flexible framework component with independent bend limiters, according to embodiments of the present disclosure.

FIG. 6 illustrates a flexible framework component with wave-matched bend limiters, according to embodiments of the present disclosure.

FIG. 7A illustrates a flexible framework component with internal wire guides, according to embodiments of the present disclosure.

FIG. 7B illustrates a flexible framework component with external wire guides, according to embodiments of the present disclosure.

FIGS. 8A-8B illustrate flexible framework components with compliant axial members, according to embodiments of the present disclosure.

FIG. 9 illustrates a flexible framework component with compliant axial members and variable-height spacing between paired bend limiters, according to embodiments of the present disclosure.

FIG. 10 illustrates a flexible framework component with an alternating neutral axis, according to embodiments of the present disclosure.

FIG. 11 illustrates a flexible framework component with axially offset undulating members, according to embodiments of the present disclosure.

FIGS. 12A-12C illustrate disks with circumferential holes for use in constructing a flexible framework, according to embodiments of the present disclosure.

FIGS. 13A-13B illustrate flexible frameworks composed of the flexible framework components discussed herein, according to embodiments of the present disclosure.

FIG. 14A illustrates a flexible framework in bending, composed of the flexible framework components, according to embodiments of the present disclosure.

FIG. 14B illustrates an alternate perspective of a flexible framework in bending, composed of the flexible framework components, according to embodiments of the present disclosure.

FIG. 15 illustrates an isometric view of a flexible framework in bending where flexible framework components are rotationally offset from one another, according to embodiments of the present disclosure.

FIG. 16 illustrates a wire-guided flexible framework, according to embodiments of the present disclosure.

FIG. 17A illustrates a wire guided flexible framework with a jacket, according to embodiments of the present disclosure.

FIG. 17B illustrates a wire guided flexible framework with a liner, according to embodiments of the present disclosure.

FIG. 18A-18D illustrate example wire guide elements with wires guides in various locations, according to embodiments of the present disclosure.

### DETAILED DESCRIPTION

The present disclosure provides flexible frameworks with neutral axis and bend limiters, in which structural wave frameworks can be used for the construction of flexible structures. The integration of a neutral axis structural members within a framework of a flexible structure defines the structure's axis of bending. Neutral axis structural members also define the axial arc length of a flexible structure in bending, and ensure the dimensional repeatability of the arc length during use. Bend limiters can be integrated into the design of the singular structural wave framework to prevent the structure being subjected to overstressed conditions. When bend limiters are fully engaged, the bend limiters define the radius of curvature for a flexible structure. Dimensions of the constraints can be designed not only to prevent overstressed conditions but also to establish a specific radius of curvature based on a specific design requirement.

In some of the figures of the present disclosure, a shared coordinate system is used (as indicated with the compasses in individual figures) with X, Y, and Z directions to aid in understanding. Although a user may orient the described devices in various positions, various dimensions may be referred to as a width, a height, or a depth, which represent measurements on the X axis, Y axis, and Z axis, respectively. The term vertical is understood to correspond to an orientation relative to the Y axis, and horizontal is understood to correspond to an orientation relative to the X axis. Furthermore, discussion of forces and moments may include directional identifiers that correspond to the same shared coordinate system and it is understood that the directional identifiers are included only to aid in understanding, and not to limit the subject matter of this disclosure.

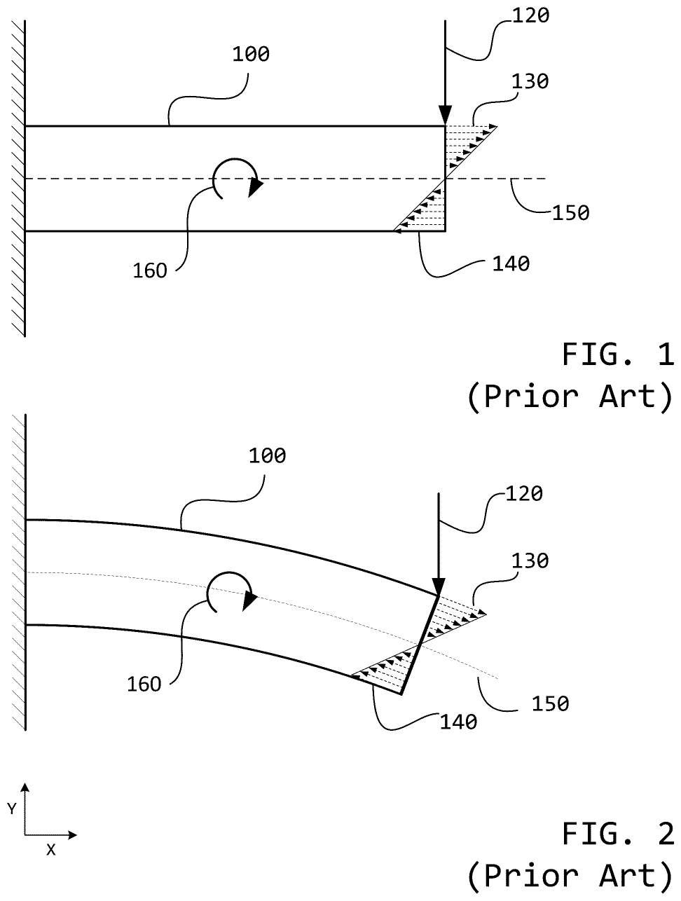

FIG. 1 is prior art, and demonstrates certain properties of elements subjected to bending stresses. FIG. 1 illustrates a cantilevered beam 100 subjected to a vertical force 120. Also illustrated are axis of bending 160 (in the Z-direction) and the tensile strains 130 and compressive strains 140 that result within the beam 100. The plane at which there are no compressive strains 140 or tensile strains 130 is the neutral axis 150, which in some cases may be referred to as the neutral surface. It has been known to establish the flexible structure wave in bending to have a radial thickness to be greater than the axial height to minimize the stress in bending. However, design-dependent sections can have both characteristics.

FIG. 2 is prior art and shows the cantilevered beam 100 of FIG. 1 where the vertical force 120 has induced bending in the cantilevered beam 100. The neutral axis 150 experiences deflection along with the beam 111, but does not experience tensile strain 130 or compressive strain 140. The plane perpendicular to the axis of bending 160 is the plane of deflection, which is an X-Y plane in the illustrated embodiment.

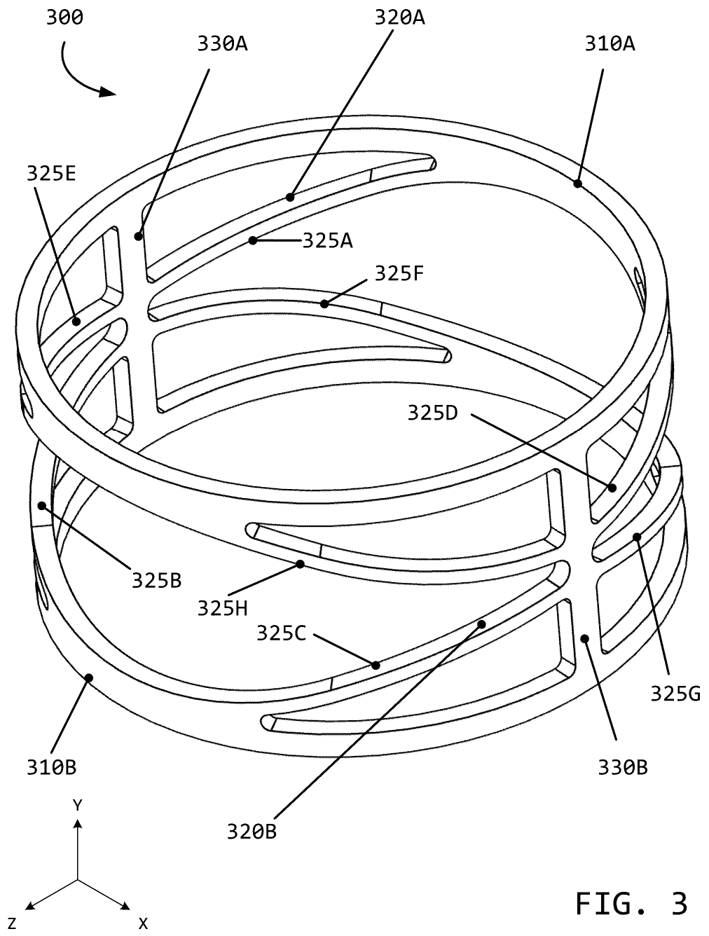

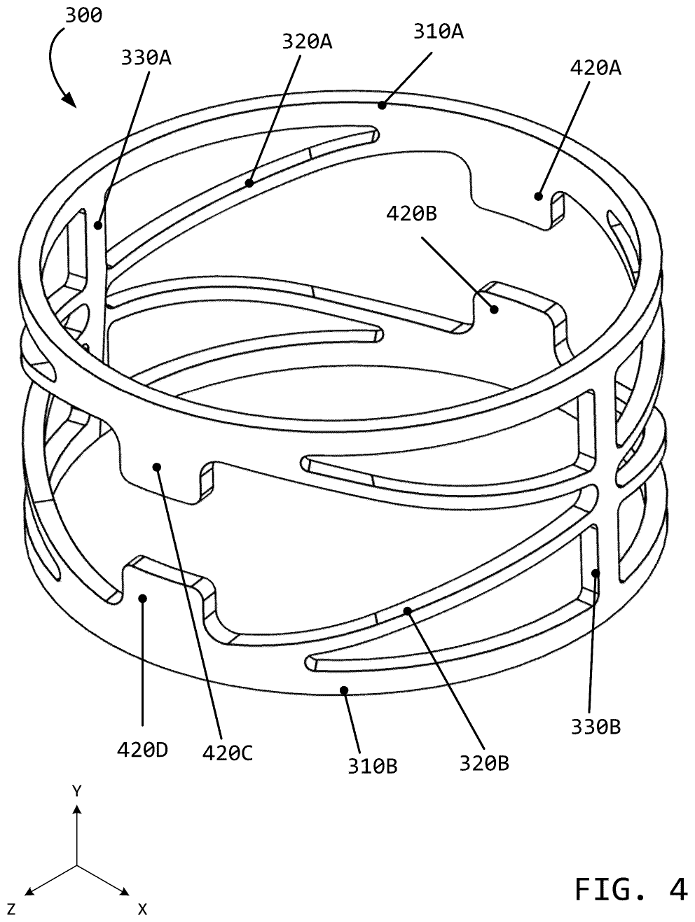

FIG. 3 shows a flexible framework component 300, having two annular members 310A-B (generally or collectively, annular members 310), two undulating members 320A-B (generally or collectively, undulating members 320), and two axial members 330A-B (generally or collectively, axial members 330), according to embodiments of the present disclosure. The annular members 310 form vertical bounds for the flexible component, and the distance between the annular members 310 is defined by the height of the axial members 330. The axial members 330 are substantially rectangular prisms, and each axial member 330 is oriented vertically (along the Y-direction), connecting on each end to opposite annular members 310. The axial members 330 form a neutral axis of deflection for the flexible framework component 300.

Although illustrated with a first axial member 330A and a second axial member 330B that are each connected to the first annular member and the second annular member diametrically opposite to one another (e.g., 180 degrees apart), the present disclosure contemplates that an uneven separation (e.g., of less than 180 degrees in one direction and more than 180 degrees in the opposing direction) is also possible. With an even (e.g., 180 degree) opposition between the axial members 330, the degree of permitted bending or deflection is equal in each direction (e.g., positive Z-ward and negative Z-ward). In the case where the axial members 330 are separated unevenly, the degree of permitted deflection will be greater in the direction of bending in which the separation is greater than 180 degrees compared to the direction of bending in which the separation is less than 180 degrees. Designs are possible with other spacing less than 180 degrees, thereby allowing a greater degree of deflection of each single cell flexible structure in the direction where the neutral axes are greater than 180 degrees. A combination of variable neutral axis locations can be used to create an overall structure of complex curves.

The undulating members 320 have an annular profile in the X-Z plane (matched with the annular members 310), and have a sinusoidal waveform defined about the circumference of the annular profile. Each undulating member 320 includes two or more full waveforms around the circumference of the undulating member 320, and may also be understood as including various arms 325A-H (generally or collectively, arms 325). The undulating members 320 are disposed between the annular members 310, and are mirrored about an X-Z plane that bisects the axial members 330. The undulating members 320 are further configured such that the waveform intersects the axial members 330 at a point where the waveform is furthest from the nearest annular member 310. In other words, the “lower” undulating member 320 intersects the axial members 330 at the apexes of the waveform, and the “upper” undulating member 320 intersects the axial members at the nadirs of the waveform. The undulating members 320 tangentially intersect the annular members 310 at points on the annular members 310 that are 90 degrees (where the Y axis is the axis of rotation) removed from the points at which the annular members 310 intersect the axial members 330.

In some examples, the undulating members 320 intersect one another at the same point that the undulating members 320 intersect the axial members 330, and in yet other examples, the undulating members 320 intersect the axial members 330 at respectively distinct points. The circumferential free length of the undulating member 320 affects the stiffness of the structure in bending. The undulating member 320 that intersects the axial member 330 and connects to the annular member 310 at a point circumferentially at 90 degrees will have greater flexibility than if connected to an annular member 310 at a point less than 90 degrees. The distance between the intersection of a first undulating member 320 and a second undulating member 320 on a given axial member 330 corresponds to the deflection stiffness of the flexible framework component 300. As the distance between intersection points is reduced, the deflection stiffness of the flexible framework component 300 increases. Adjusting the distance between the intersections of a first undulating member 320 and a second undulating member 320 on a given axial member 330 can be achieved by either changing the length of the axial members 330, and as a result, the height of the flexible framework component 300; or, by adjusting the amplitude of the waveform of the undulating members 320. An increased amplitude of the waveform of the undulating member 320 results in closer undulating-axial intersections, and thus an increased deflection stiffness.

Both the undulating members 320 and the axial members 330 are defined within the profile of the annular members in the X-Z plane. Stated differently, the flexible framework component 300 forms a skeleton of a hollow cylinder.

The flexible framework component 300 is configured to deflect in a predictable and dimensionally repeatable manner when various stresses are applied. The axial members 330 contain neutral axes, and define the axis of bending of the flexible framework component 300. For example, when a force is applied to the flexible framework component 300 in the Y-direction, the resulting bending occurs about the X-axis rotation. Any force acting on the flexible framework component 300 having components in the Y-direction (with the exception of forces coaxial with the neutral axis) will result in deflection where the axis of bending is in the X-direction. In other words, the placement of the neutral axis defines the axis of rotation of the flexible framework component.

In addition to providing neutral axes, the axial members 330 provide resistance to axial forces, and prevent the flexible framework component 300 from compressing or extending in length when subjected to an axial load. The compression resistance provided by the axial members 330 also ensures a constant axial arc length of the flexible framework component 300 when in bending.

According to some embodiments, flexible framework components 300 are constructed by additive manufacturing, using methods such as 3-D printing. In such embodiments, flexible framework components 300 are constructed of thermoplastics, resins, polymers, or metals.

According to some embodiments, flexible framework components 300 are constructed by removing material from an existing hollow cylinder, such as a section of pipe or tubing. In such embodiments, flexible framework components 300 are constructed of plastics, polymers, metals, and the like. The present disclosure further contemplates embodiments formed by chemical etching, laser etching, and laser cutting.

FIG. 4 shows an isometric view of a flexible framework component 300 with dependent bend limiters 420, according to embodiments of the present disclosure. The flexible framework component 300 with dependent bend limiters 420 has two annular members 310, two undulating members 320, and two axial members 330; and further includes four dependent bend limiters 420A-D (generally or collectively, dependent bend limiters 420). The dependent bend limiters 420 are protrusions that emanate from the annular members 310 towards one another along the Y-direction, and are 90 degrees removed from the axial members 330, at the tangential intersection of the undulating members 320 and annular members 310. The illustrated flexible framework component 300 has four dependent bend limiters 420 divided into two pairs, where each dependent bend limiter 420 is directed towards the dependent bend limiter 420 protruding from the opposite annular member 310.

The dependent bend limiters 420 are disposed within the plane of deflection of the flexible framework component 300 with dependent bend limiters 420, and are configured such that when bending occurs (axis of bending in the Z-direction) the dependent bend limiters 420 will contact one another and prevent further deflection. In some examples, dependent bend limiters 420 prevent plastic deformation of the material from which the flexible framework component 300 is constructed. The dependent bend limiters 420 can be configured to prevent permanent deformation and overstressing of the flexible framework component 300 when included thereon.

The dependent bend limiters 420 can be described as bending stops, which contact one another when a certain amount of deflection occurs. By varying the length of the dependent bend limiters 420, the user can tune the maximum deflection of a given flexible framework component 300 with dependent bend limiters 420. By increasing the length of each dependent bend limiter 420, contact occurs after less deflection, resulting in a decreased maximum deflection, and by decreasing the length of each bend limiter 420, contact occurs after more deflection, resulting in an increased maximum deflection. In other words, the length of the dependent bend limiters 420 is inversely related to the maximum deflection of the flexible framework component 300 with dependent bend limiters 420.

Although illustrated with four dependent bend limiters 420 in FIG. 4, the present disclosure contemplates that a flexible framework component 300 may have three dependent bend limiters (e.g., 420A-C), two opposing dependent bend limiters (420A-B), two un-opposing bend limiters emitting from the same annular member (e.g., 420A and 420C from 310A), two un-opposing bend limiters emitting from the different annular members (e.g., 420A from 310A and 420D from 310B), or one bend limiter (e.g., 420A). Additionally, although illustrated as substantially uniform to one another, the present disclosure contemplates that the relative sizes of the dependent bend limiters 420 may differ from one another to affect different flexing properties in a given flexible framework component 300.

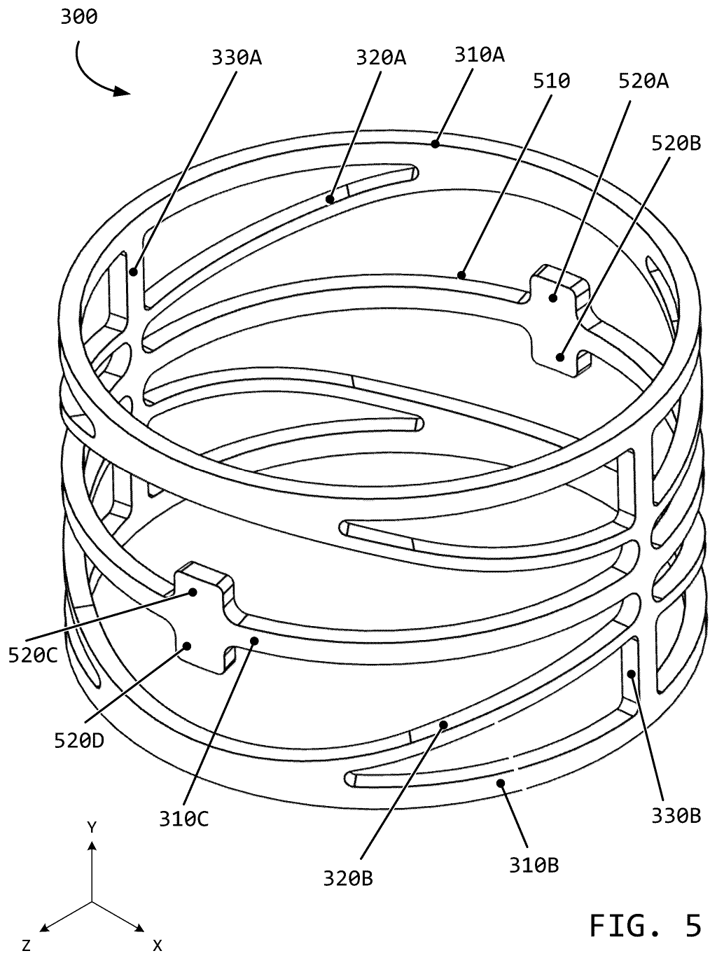

FIG. 5 shows a flexible framework component 300 with independent bend limiters 520A-D (collectively or generally, independent bend limiters 520), according to embodiments of the present disclosure. As in previously described examples, flexible framework components 300 with independent bend limiters 520 include two annular members 310, two undulating members 320, and two axial members 330. A flexible framework component 300 with independent bend limiters 520 and further includes third annular member 310C, disposed between the undulating members 320. The independent bend limiters 520 emanate from the third annular member 310C, above and below in the Y-direction, towards the first two annular members 310A-B. The independent bend limiters 520 function much the same as the dependent bend limiters 420, excepting that as deflection occurs in the flexible framework component 300, the independent bend limiters 520 contact the annular members 310, preventing further deflection.

Although illustrated with four dependent bend limiters 520 in FIG. 5, the present disclosure contemplates that a flexible framework component 300 may have three dependent bend limiters (e.g., 520A-C), two opposing dependent bend limiters (520A-B), two un-opposing bend limiters emitting from the same side of the third annular member (e.g., 420A and 420C from 510), two un-opposing bend limiters emitting from the different side of the third annular member (e.g., 520A and 520D from 510), or one bend limiter (e.g., 520A). Additionally, although illustrated as substantially uniform to one another, the present disclosure contemplates that the relative sizes of the dependent bend limiters 520 may differ from one another to affect different flexing properties in a given flexible framework component 300.

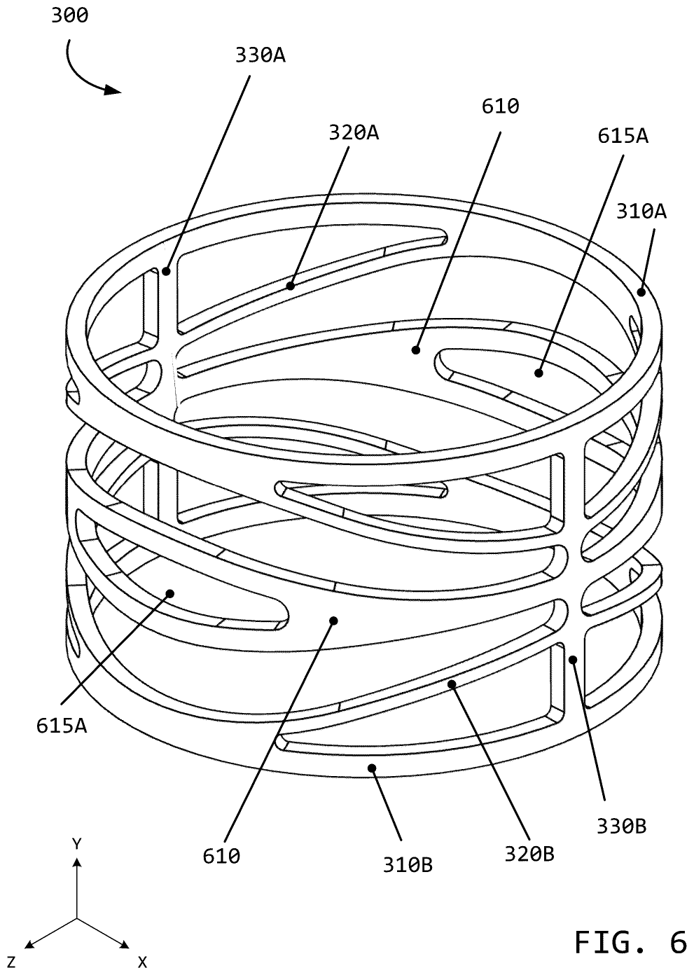

FIG. 6 illustrates a flexible framework component 300 with a wave-matched bend limiter 610, according to embodiments of the present disclosure. The wave matched bend limiter 610 is an annular component disposed between the undulating members 320, and intersects the axial members 330. The wave-matched bend limiter matches the curvature of the waveform of the undulating component, such that when the flexible framework component deflects, the undulating components contact the wave-matched bend limiter to prevent further deflection. The wave-matched form of the wave-matched bend limiter allows for a greater surface area of the contact area between an undulating component and the wave matched bend limiter, as the contact occurs over a larger edge of the wave matched bend limiter 610 and undulating member 320. The larger contact area serves to distribute force over a larger volume of the bend limiter, allowing for greater compressive forces which may incur deformation in, a comparable smaller, dependent bend limiter 420 or an independent bend limiter 520.

In some embodiments, such as the illustrated embodiment, the wave-matched bend limiter 610 incudes a cut-out sections 615A-B (generally or collectively, cut-out sections 615) proximate to the plane of deflection. The cut-out sections 615 provide the wave-matched bend limiters 610 with compressibility, allowing for deflection after contact between the wave-matched bend limiters 610 and the undulating members 320, albeit at an increased resistance, relative to the deflection resistance prior to the contact.

In some embodiments, the wave-matched bend limiter does not include cut-out sections 615, and deflection of the flexible framework component is ceased after contact between the wave-matched bend limiter 610 and the undulating members 320.

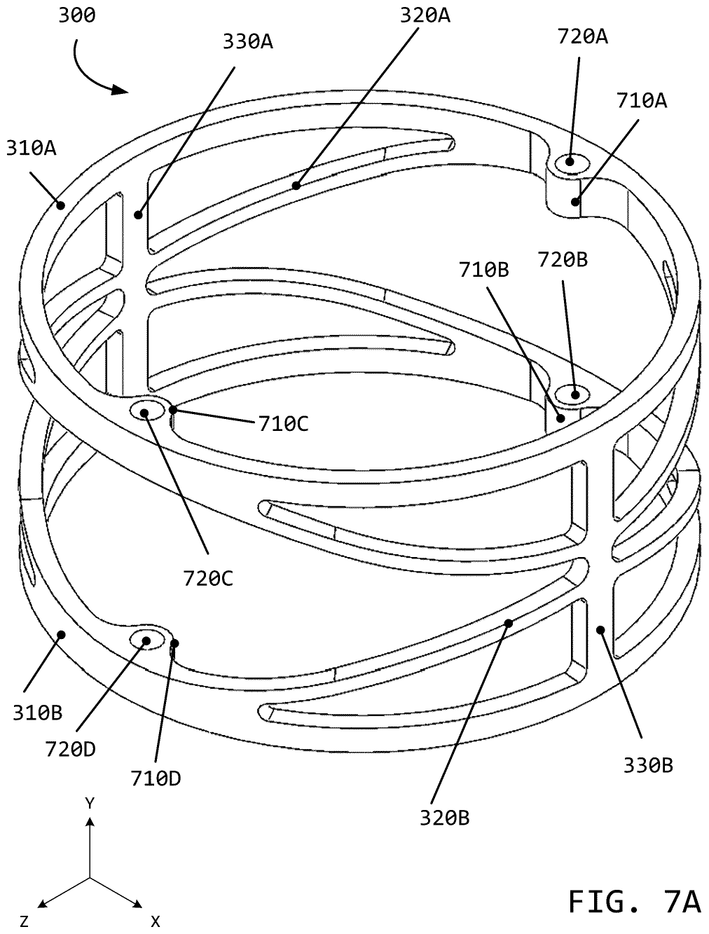

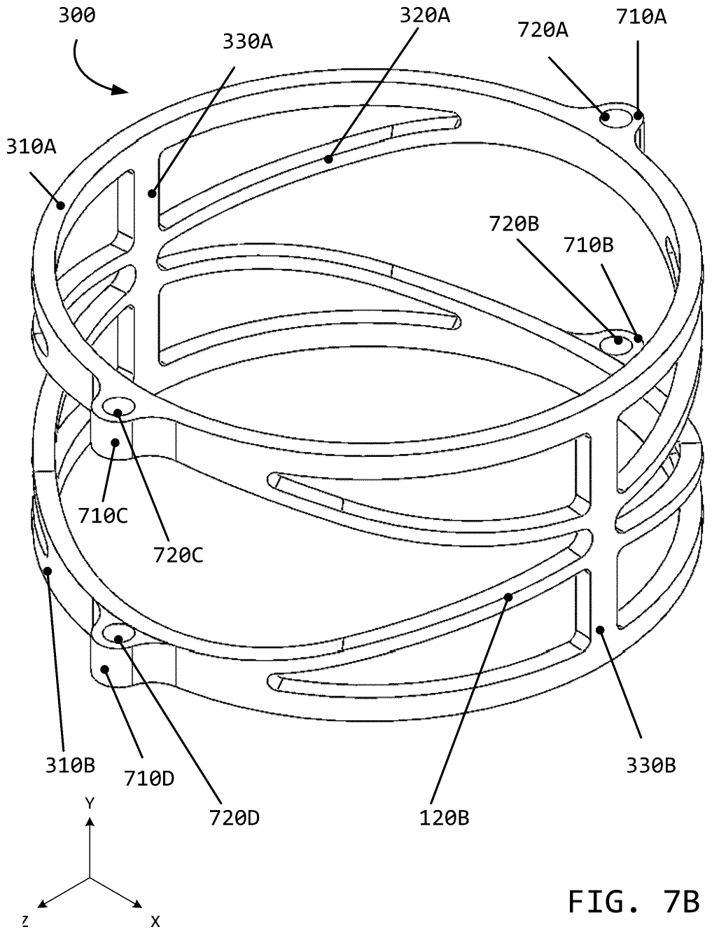

FIG. 7A illustrates a flexible framework component 300 with guide holes 720A-B (generally or collectively, wire guide holes 720) disposed on the interior of the annular member 310, according to embodiments of the present disclosure. In some examples, the flexible framework component 300 includes protrusions 710 on the outer circumference of the annular members 310, each of which defines a corresponding guide hole 720A-D. The protrusions 710, and guide holes 720 are 90 degrees removed from the axial members 330 and protrude in the Z-direction, towards the center of the annular member 310. Guide holes can be round, square, rectangular, or any other appropriate shape as needed per the design.

FIG. 7B illustrates a flexible framework component 300 with guide holes 720A-D disposed on the exterior of the annular members 310, according to embodiments of the present disclosure. The flexible framework component 300 includes protrusions 710A-D on the outer circumference of the annular members 310, each of which defines a corresponding guide hole 720A-D. The protrusions 710, and guide holes 720 are 90 degrees removed from the axial members 330 and protrude in the Z-direction, towards the center of the annular member 310.

Although illustrated with four protrusions 710 and guide holes 720 in FIGS. 7A-B, the present disclosure contemplates that a flexible framework component 300 may have one protrusion 710A, two protrusions 710A-B, three protrusions 710A-C, or four protrusions 710A-D, each having one or more guide hole 720. Additionally, although illustrated as substantially uniform to one another, the present disclosure contemplates that the relative sizes of the protrusions 710 and guide holes 715 may differ from one another to affect different joining and attachment properties in a given flexible framework component 300.

In some embodiments, the Y-directional height of a protrusion is not greater than the Y-directional height of an annular member 310, and in some embodiments, the Y-directional height of a protrusion 710 is equal to the Y-directional height of an annular member 310.

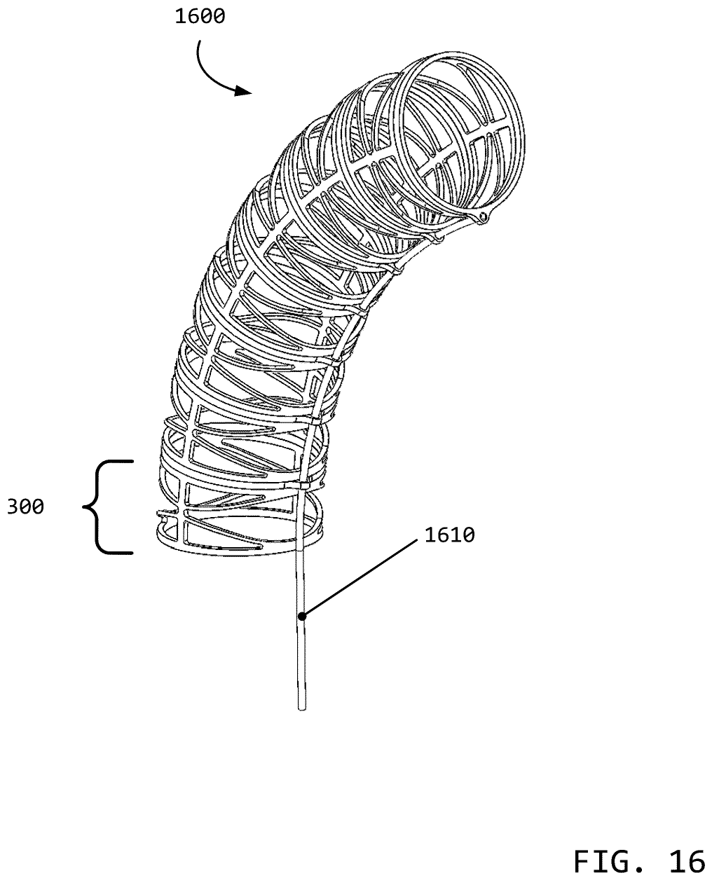

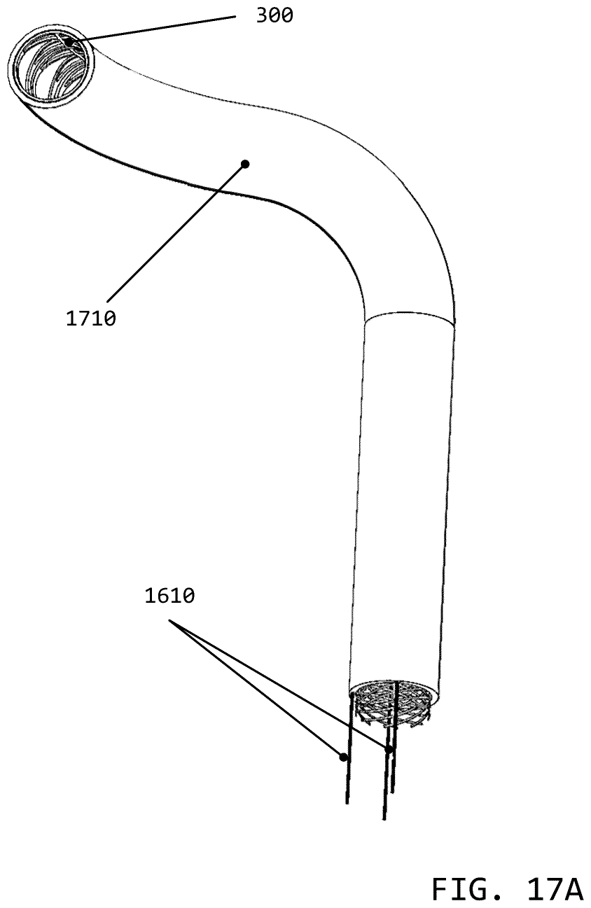

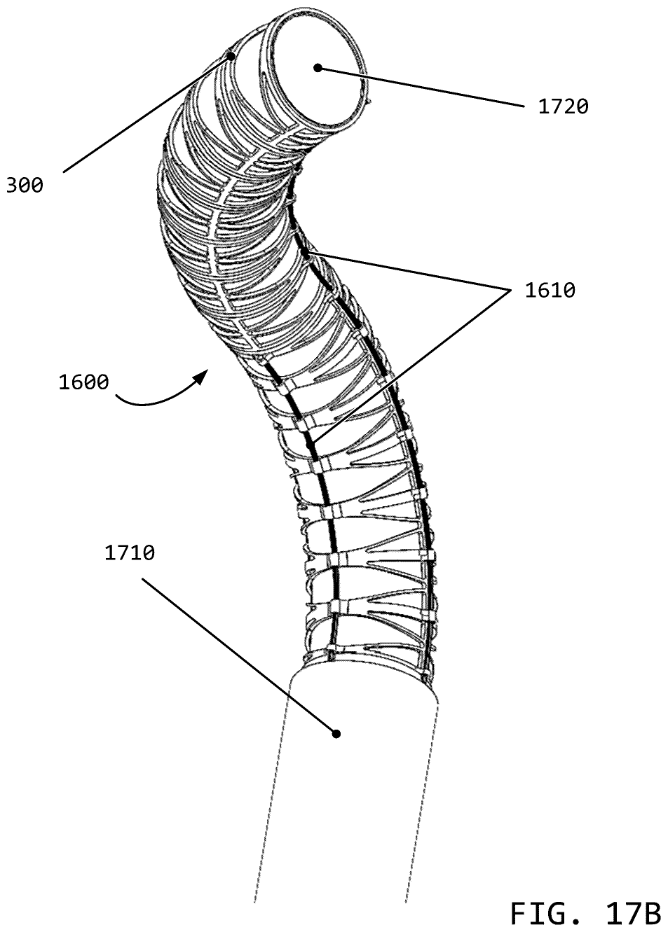

In some embodiments, the protrusions 710 and guide holes 720 are configured to accommodate guide wires, which, when tensioned and secured, can provide the forces required to induce bending and deflection in flexible framework component 300 with guide holes 720. Further discussion of guide wires and related systems can be found in FIGS. 16 and 17A-17B and the associated description.

In some examples the protrusions 710 and guide holes 720 are configured to accommodate fasteners or adhesives, which can be used to secure multiple flexible framework components 300 with guide holes 715 together. Such fasteners may include bolts, screws, rivets, pins and the like. In other examples, the protrusions 710 do not include guide holes 720 and simply provide surface area by which to adhere multiple flexible framework components 300 together. Such processes may include the use of welding, heat bonding, brazing, and chemical adhesives.

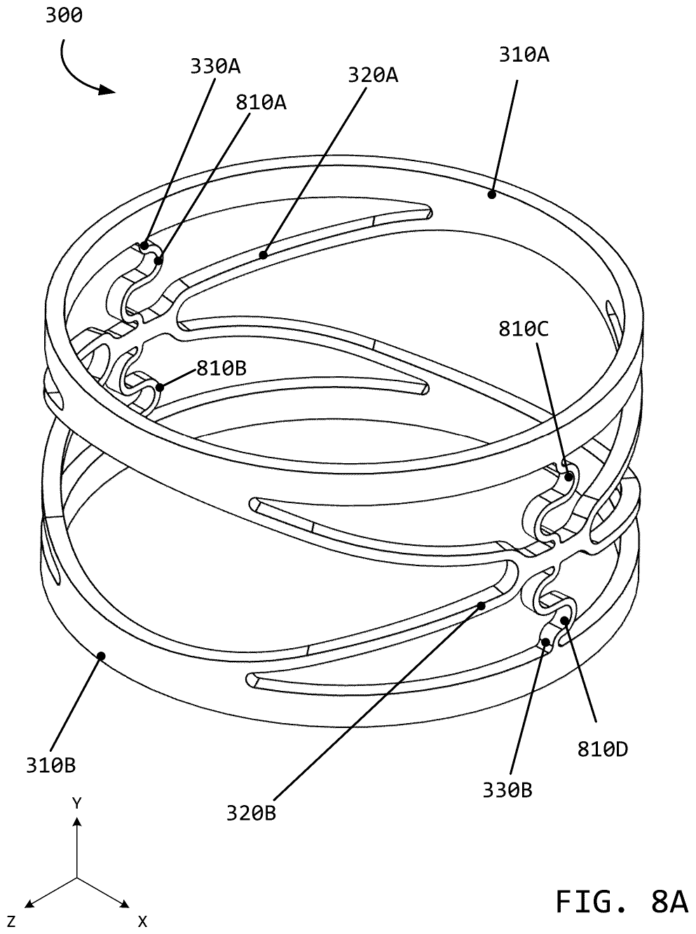

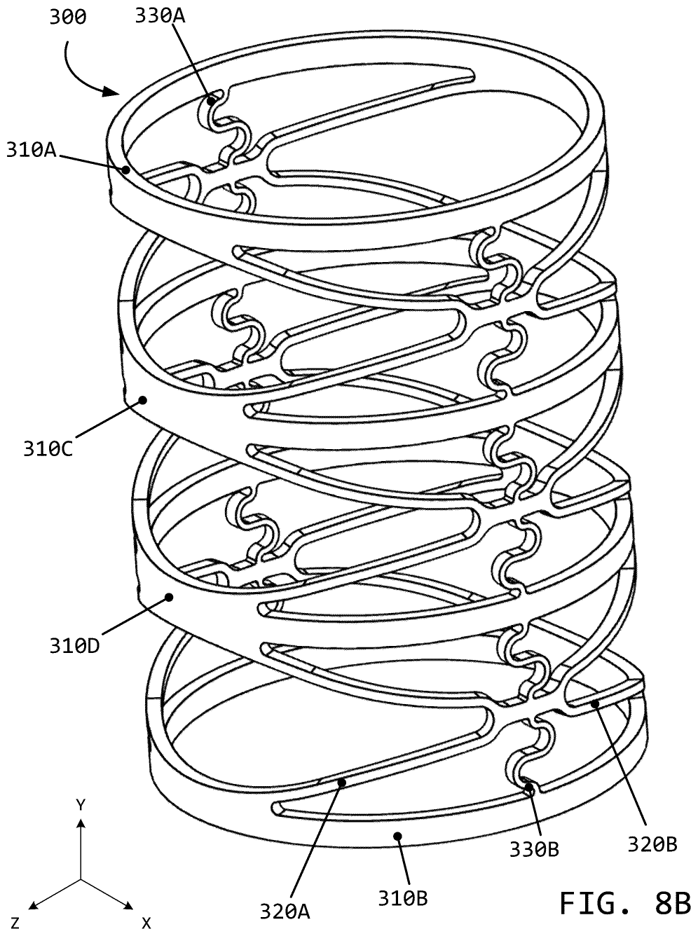

FIGS. 8A-8B illustrate flexible framework components 300 with compliant axial members 330, according to embodiments of the present disclosure. Axial members 330 may be generally in-line with the longitudinal axis of a flexible framework (e.g., substantially aligned with the Y-axis of the flexible framework component 300 when in an un-bent configuration) as illustrated in FIGS. 3-11, or may include two or more compliant structures 810A-L (generally or collectively, compliant structures 810) that can provide greater structural flexibility in bending compared to in-line axial members 330 by absorbing some of the stress from the structural wave. Indeed, the integration of a compliant axial members 330 within a framework has been found to reduce the restriction in bending in the axis 90 degrees to the primary axis of bending, while being sufficiently supportive to influence the primary axis of bending. By including compliant structures 810, the axial members 330 thereby allow a flexible framework design to exhibit further movement or progression of the flexible framework through a vessel or pathway than an otherwise similar design using in-line axial members 330.

The compliant structures 810 define an undulating pathway between two connection points within a given flexible framework component 300, such as an annular member 310 or the intersection of the undulating members 320. Generally, the connection points for the compliant structures 810 remain in-line with one another on a given side of the flexible framework component 300, with each axial member 330 being disposed about 180 degrees apart from each other relative to the connection points. As illustrated, each compliant structure 810 is defined with curves in the same direction as corresponding compliant structure 810 at the same height (e.g., along the Y-axis), such that the bottommost undulation in the first axial member 330A and the bottommost undulation in the second axial member 330B are both defined with a counterclockwise curve, and similarly the second-bottommost undulation in the first axial member 330A and the second-bottommost undulation in the second axial member 330B are both defined with a clockwise curve. The present disclosure contemplates that the curves as the same heights in opposing compliant structures may instead be counter-curved so that, for example, for each clockwise curve in a first axial member 330A, an opposing counterclockwise curve is defined at the same height in the second axial member 330B.

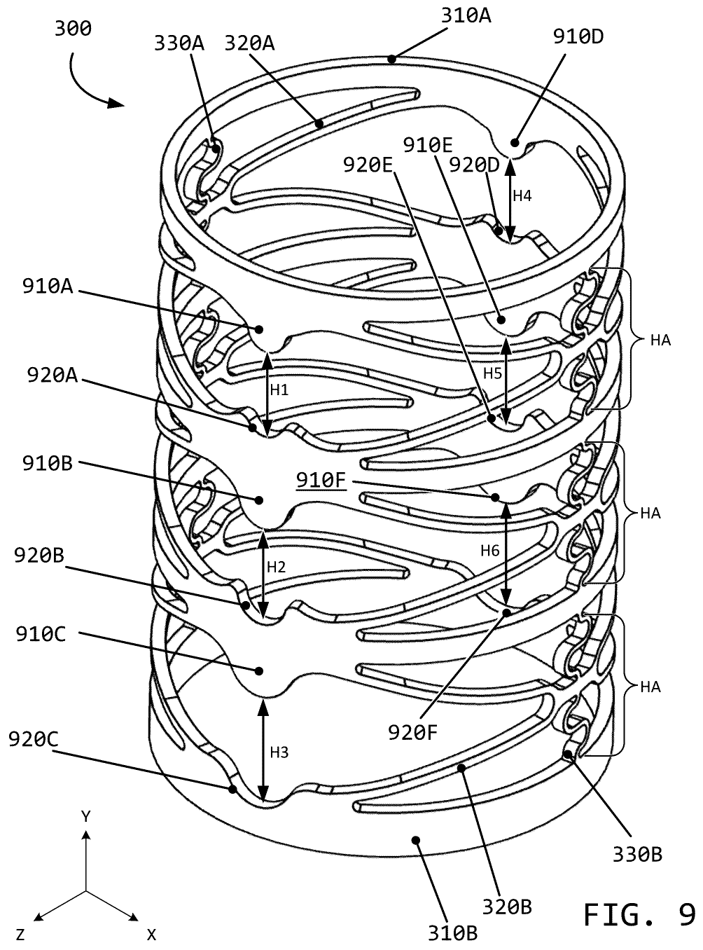

Although illustrated with generally evenly sized S-shaped compliant structures 810, the present disclosure contemplates that compliant structures 810 can take other shapes and be provided in various numbers and orientations. For example, FIG. 8A illustrates an embodiment with four compliant structures 810A-D (two per axial member 330), while FIG. 8B illustrates an embodiment with twelve compliant structures 810A-L (six per axial member 330). In another example, the compliant structures 810 can define uneven S-shapes, with one end or undulation of a compliant structure 810 larger than the other. Similarly, although illustrated in FIGS. 8A-9B with substantially semi-circular undulations in a compliant structure 810, the undulations may be have different arcuate shapes, or generally define a Z-like shape. In another example, although illustrated with two undulations per compliant structure 810 (one curved clockwise and one curved counterclockwise), a compliant structure 810 may be defined by one undulation or by more than two undulations in the plane defined by the outer circumference of the annular members 310 such that the compliant structure 810 defines one or more gaps in material (e.g., air gaps) along a straight-line path between the annular members 310 in the height direction (e.g., the Y-axis).

FIG. 9 illustrates a flexible framework component 300 with compliant axial members 330 (e.g., including compliant structures 810) and variable-height spacing between paired bend limiters, according to embodiments of the present disclosure. The paired bend limiters include positive protrusions 910A-F (generally or collectively, positive protrusions 910) and negative protrusions 920A-F (generally or collectively, negative protrusions 920) that are located and shaped to form a corresponding pair to contact one another when the flexible framework component 300 is bent to thereby control a degree of bending permitted by the flexible framework component 300. Each of the protrusions (positive and negative) is defined with respect to an annular member, approximately 90 degrees from the axial members 330 on either side. The positive protrusions 910 project from the annular members 310 (e.g., upward or downward in the Y-direction) and the negative protrusions 920 are defined in the material of the annular members 310 to define an accommodating space for the positive protrusions to seat into when the flexible framework component 300 bends, to thereby control an extent of bending permitted. Although illustrated with four annular members 310A-D with associated spacing, the present disclosure contemplated that variable-height spacing may be achieved in flexible framework components 300 having fewer than or more than four annular members 310A-D.

The size and shape of the paired bend limiters are varied in the current example, such that a first height H1 between the first pair (910A/920A) is different from the second height H2 between the second pair (910A/920A), and different from the third height H3 between the third pair (910C/920C) (e.g., H1≠H2≠H3), despite the height between each pair of neighboring annular members 310 being the same (e.g., an inter-annular height HA). In some examples, a subset of the spacings between the paired bend limiters on a given side of the flexible framework component 300 are the same height (e.g., H1=H2≠H3). In some examples, one or more (but not all) of the spacings between the paired bend limiters on a given side of the flexible framework component 300 are the same height as the inter-annular height HA (e.g., H2=HA≠H1).

Although the illustrated example is shown with three pairs of bend limiters on either side of the flexible framework component 300 with equivalently sized heights between opposing pairs of bend limiters at the same height (on the Y axis) of the flexible framework component 300, the present disclosure contemplates that other relationships are possible. For example, as illustrated, the first height H1 between the first pair (910A/920A) is the same as the fourth height H4 between the fourth pair (910D/920D), the second height H2 between the second pair (910B/920B) is the same as the fifth height H5 between the fifth pair (910E/920E), and the third height H3 between the third pair (910C/920C) is the same as the sixth height H6 between the sixth pair (910E/920E). However, the opposing heights in some embodiments are non-equivalent in the opposing pair of bend limiters, to thereby affect a non-uniform extent of permitted bending for the flexible framework component 300. In some examples, the heights are mirrored along the Y-axis (e.g., H1=H6, H2=H5, H3=H4, where H1≠H3). In some examples, the bend limiters are omitted from one side of the flexible framework component 300 (e.g., H4=H5=H6=HA).

The present disclosure contemplates that the individual heights may define different equivalences to one another from the illustrated example, and that one or more of the heights between a pair of bend limiters may be equal to the height between the annular members 310 (e.g., H1=HA, H2=HA, etc.).

Each of the heights discussed herein shall be understood to refer to the distance in the Y-direction (e.g., the longitudinal axis) of the flexible framework component 300 when in an un-bent state. When referring to the distance between two bend limiters, the distance shall be understood to be measured from the location of maximal projection away from (or into, in the case of a negative protrusion 920) the respective annular member 310.

Bend limiters can be integrated into the design of the singular structural wave framework to prevent the structure from being subjected to overstressed conditions. When bend limiters are fully engaged, the bend limiters define the radius of curvature for a flexible structure. Dimensions of the constraints can be designed not only to prevent overstressed conditions, but also to establish a specific radius of curvature based on a specific design requirement.

Bend limiters integrated into the design with equal heights will form a geometric shape of a circle (a given radius and circumferential length) based on the number of equal length wave cells. The integration of bend limiters of variable heights along the length of the structure provides a way of creating other radial geometric shapes being parabolic and hyperbolic when fully engaged. The present disclosure contemplates that a designer can integrate bend limiters with a mix of variable heights and fixed heights to design complex geometric shapes in a single plane.

Additionally, flexible structures during use may be subjected to torsional loads during use—seeking to bend or twist the structure in a direction other than the intended axis for bending. The bend limiters as a result of this torsional loading may be displaced and lose the compressive contact possibly resulting in an overstressed condition. However, when using paired positive and negative bend limiters with conformal shapes, the design offers a locking engagement between the pair, in which the locking bend limiters, when engaged, prevent or reduce the bend limiter (and thereby the structure) from being displaced by torsional loading. The locking bend limiters also provide a structural axis alignment feature. When a flexible structure is in bending the alignment of the bend limiter can be influenced by outside forces or loading deflecting the axis of bending. The nesting characteristic of the locking bend limiters provides a way to achieve axial alignment when outside forces cause the axis to bend out of plane.

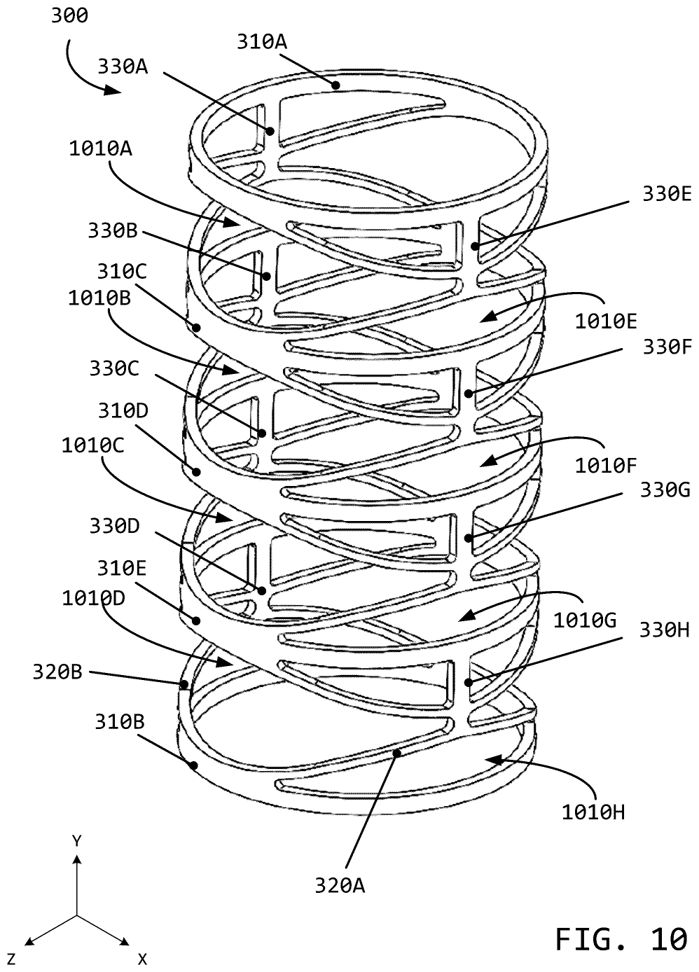

FIG. 10 illustrates a flexible framework component 300 with an alternating neutral axis, according to embodiments of the present disclosure. The illustrated flexible framework component 300 includes five annular members 310A-E, two undulating members 320A-B, eight axial members 330A-H, in which the axial members 330 do not extend for the entire height (e.g., in the Y-direction), but instead define a plurality of neutral axis gaps 1010A-H (generally or collectively, neutral axis gaps 1010) in which material is omitted. The neutral axis gaps 1010 permit (constrained) bending in a direction other than the main direction of bending (e.g., from the Y-axis towards the X-axis in addition to from the Y-axis towards the Z-axis), and provide additional ease of bending in the main direction of bending by reducing the amount of material to bend when bending the flexible framework component 300.

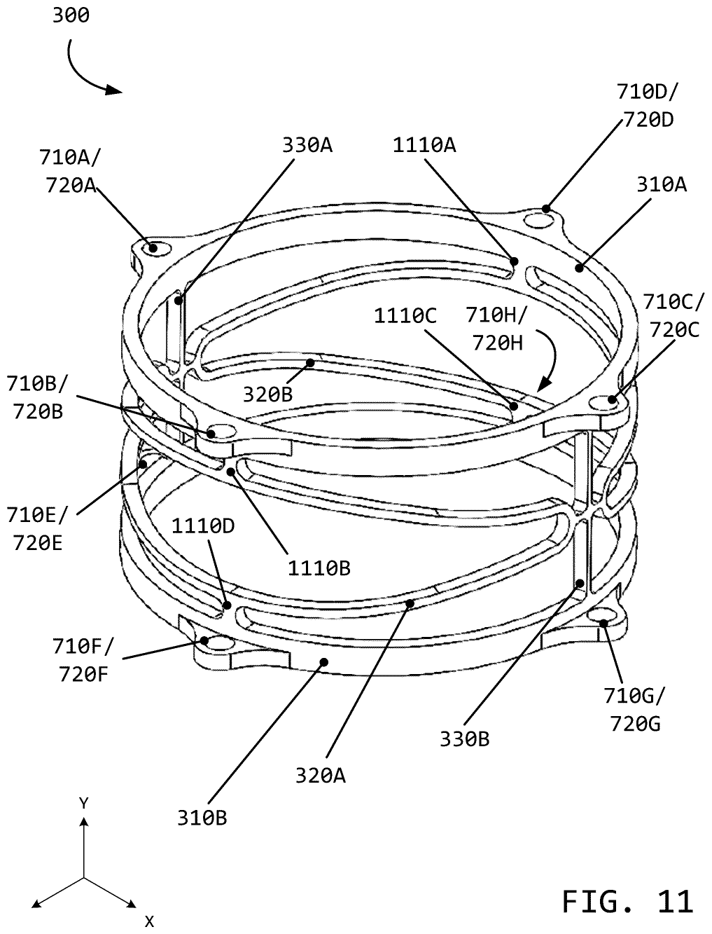

FIG. 11 illustrates a flexible framework component 300 with axially offset undulating members 320, according to embodiments of the present disclosure. The illustrated flexible framework component 300 includes a two annular members 310A-B, two undulating members 320A-B, two axial members 330A-B, and a plurality of protrusions 710A-H on the outer circumference of the annular members 310, each of which defines a corresponding guide hole 720A-H (protrusion 710H and guide hole 720H being obscured in the view of FIG. 11), although a greater number of components may be incorporated into a flexible framework component 300 using the various features discussed in the present disclosure. As illustrated, a first subset of the protrusions 710 (710A, 710C, 710E, 710G) are circumferentially aligned with the axial members 330 of the flexible framework component 300, while a second subset of the protrusions are aligned 90 degrees offset from the neutral axis defined by the axial members 330, and are instead circumferentially aligned with axial offsets 1110A-D (generally or collectively, axial offsets 1110) associated with the undulating members 320.

The axial offsets 1110, when included in the design of a flexible framework component 300, connect between an annular member 310 and an undulating member 320, which extend in a direction equivalent to the axial members 330 (e.g., the Y direction), albeit at a rotational offset (e.g., 90 degrees) from the neutral axis defined by the axial members 330. The vertical offset from the annular members 320 imparted by the axial offsets 1110 to the undulating members 320 imparts additional flexibility to the flexible structural component 300; allowing for easier (e.g., with a lower applied force) or greater ability to bend (e.g., from the Y-axis towards the Z-axis) than designs that do not axial offsets 1110.

The present disclosure contemplates that, all else being equal, that designs with “taller” axial offsets 1110 generally offer a greater increase in flexibility that designs with “shorter” axial offsets 1110, and that a designer can tune the total height of the axial offsets to affect the flexibility of the component 300. However, the benefits of added flexibly begin to decrease when the total height of the axial offsets 1110 occupy more than 50% of the total distance between the annular members. Accordingly, the height of a given axial offset 1110 is generally recommended to be between 1% and 20% of the distance between the associated annular members 310. The present disclosure contemplates that the axial offsets 1110 may extend from the annular members 310 for a same distance as one another, or may extend from the annular members 310 for variable distances (e.g., axial offset 1110A and axial offset 1110B extend for a first distance versus axial offset 1110C and axial offset 1110D, which extend for a second distance).

Although illustrated with axial offsets included on both an upper and lower annular member 310, the present disclosure contemplates that one annular member 310 may include, and one annular member 310 may omit axial offsets 1110, with the undulating members 320 directly connected to the annular member 310. For example, the second annular member 310B may omit the third axial offset 1110C and the fourth axial offset 1110D, while the first annular member 310A includes the first axial offset 1110A and the second axial offset 1110B.

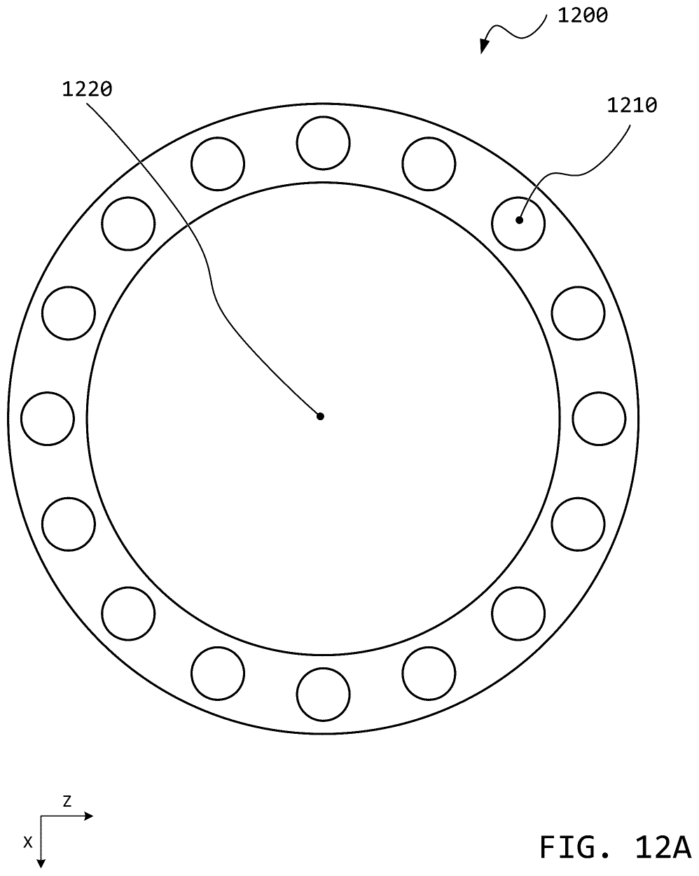

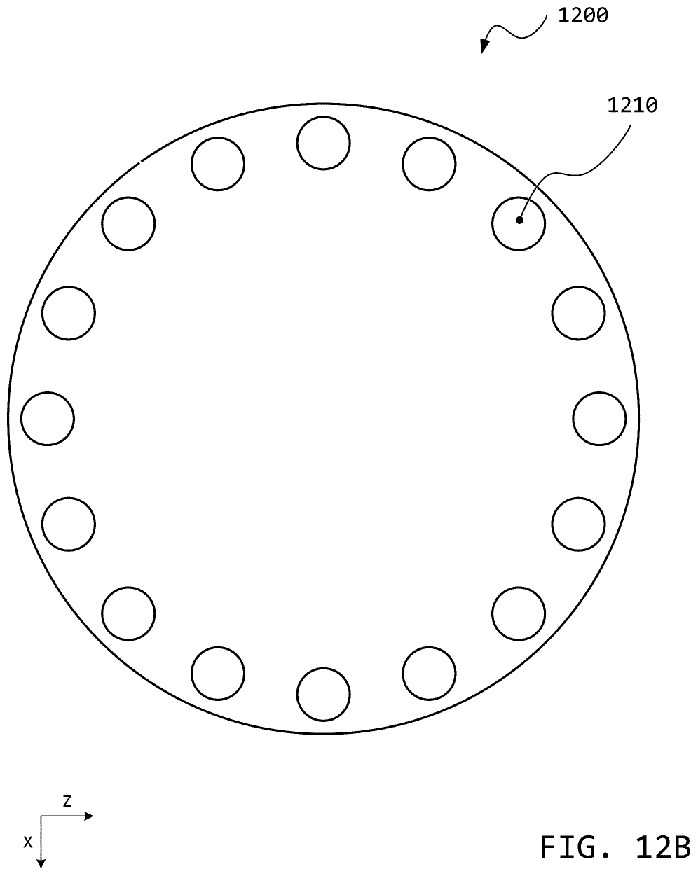

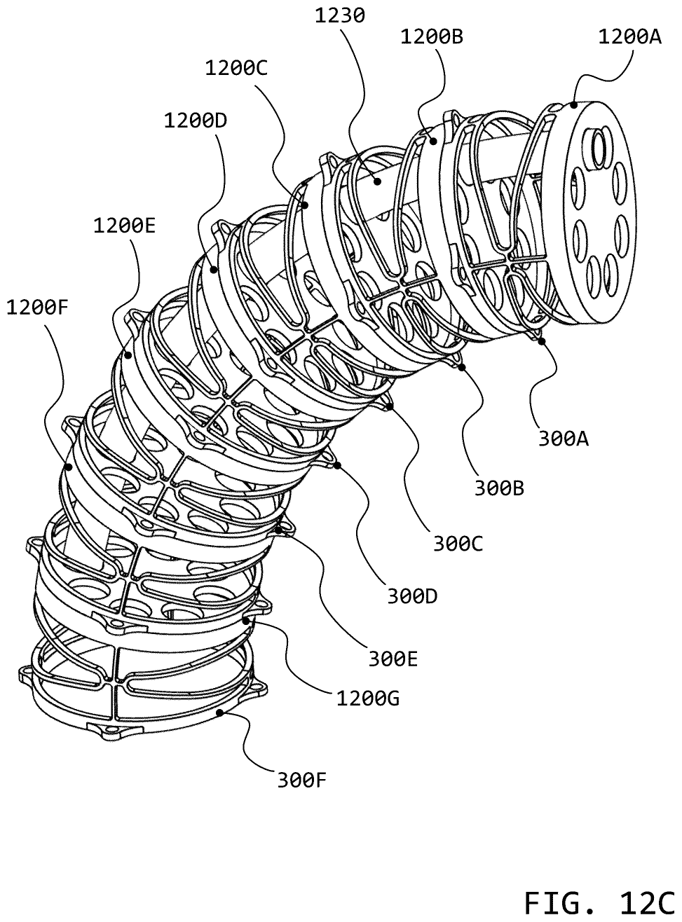

FIGS. 12A-12C illustrate disks 1200, which may be integrated into a structure using flexible framework components 300 to accept various guidewires, flexible electrical wires, flexible optical strands, conductors, conduits, specific pathways, or the like, according to embodiments of the present disclosure.

FIG. 12A illustrates a disk 1200 having circumferential holes 1210 the center points of which are located proximately to the edge of the disk 1200, and a central hole 1220, having a shared center point with the disk 1200. The disk 1200 is a rigid circular plate, having the same radial dimensions as that of the flexible framework component 300. The illustrated embodiment is a disk 1200 containing sixteen circumferential holes 1210, but the present disclosure further contemplates embodiments having no circumferential holes 1210 in addition to embodiments having about more or fewer circumferential holes 1210 than illustrated (e.g., about five, ten, twenty, or fifty circumferential holes 1210). The diameters of the circumferential holes 1210 are illustrated as equivalent, but embodiments having variably distinct diameters of circumferential holes 1210 are contemplated.

Similarly, FIG. 12B illustrates disk 1200 having circumferential holes 1210 the center points of which are located proximately to the edge of the disk 1200, but omits a central hole 1220. Although illustrated in FIGS. 12A and 12B as separate elements from a flexible framework components 300, which a manufacturer can secure between two neighboring flexible framework components 300 in a structure, the present disclosure contemplates that one or more of the ring members 310 of a flexible framework component 300 may incorporate a disk 1200 as an integral element.

According to some embodiments, some of the circumferential holes 1210 are configured to align with the guide holes 715 of flexible framework components 300 with guide holes 720. In some embodiments, a disk 1200 is disposed in between two flexible framework components 300 in order to form a flexible framework, or integrally manufactured with one or more flexible framework components in the flexible framework.

FIG. 12C illustrates a flexible framework that incorporated disks 1200a-h into the design, according to embodiments of the present disclosure. According to some embodiments, one or more of the circumferential holes 1210 and the central hole 1220 are configured to accommodate various flexible conduits 1230, which may include guidewires, flexible electrical wires, flexible optical fibers, conductors, tubes, conduits, specific pathways, or the like, and insulative or protective elements therefor. In such an embodiment, when multiple disks 1200 are integrated into a flexible framework, a flexible conduit 1230 can be inserted through the central holes 1220 of multiple disks 1200 and a fluid, electrical, or optical transportation system can be implemented within the flexible framework.

In various embodiments, one or more disks 1200 are placed between flexible framework components 300 to act as washers or friction-reducing members, provide an adhesion point between neighboring flexible framework components, or provide additional mounting points or guides via the circumferential holes 1210 for various accessories or guidewires. In some embodiments, a disk 1200 is mounted at the end or between neighboring flexible framework components 300 to provide a cap or a stop to reduce fluid flow, or define a continuous pathway through the entire length of the structure while in bending, or improve sterility of the device while in use.

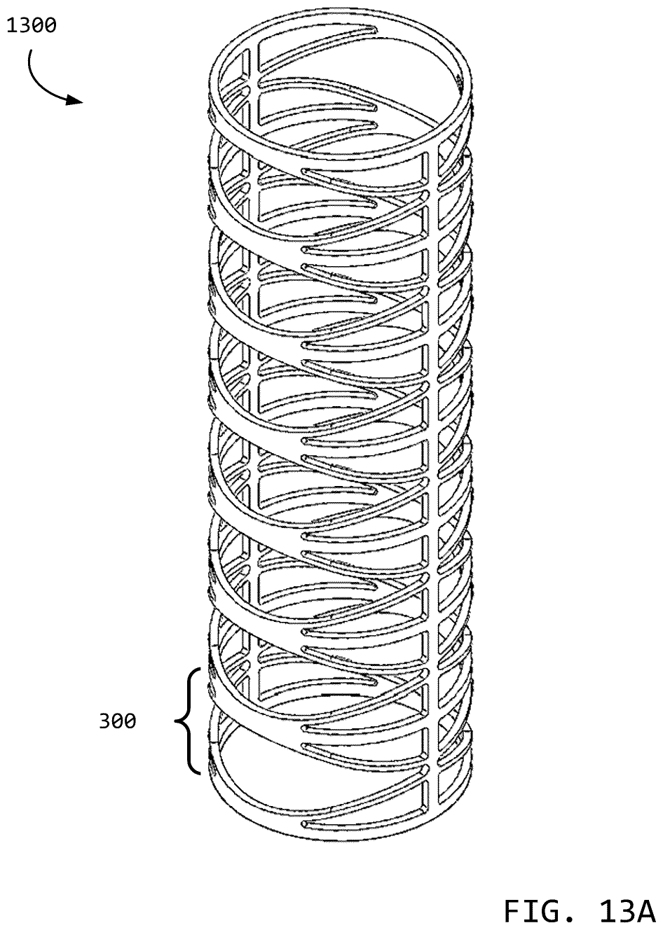

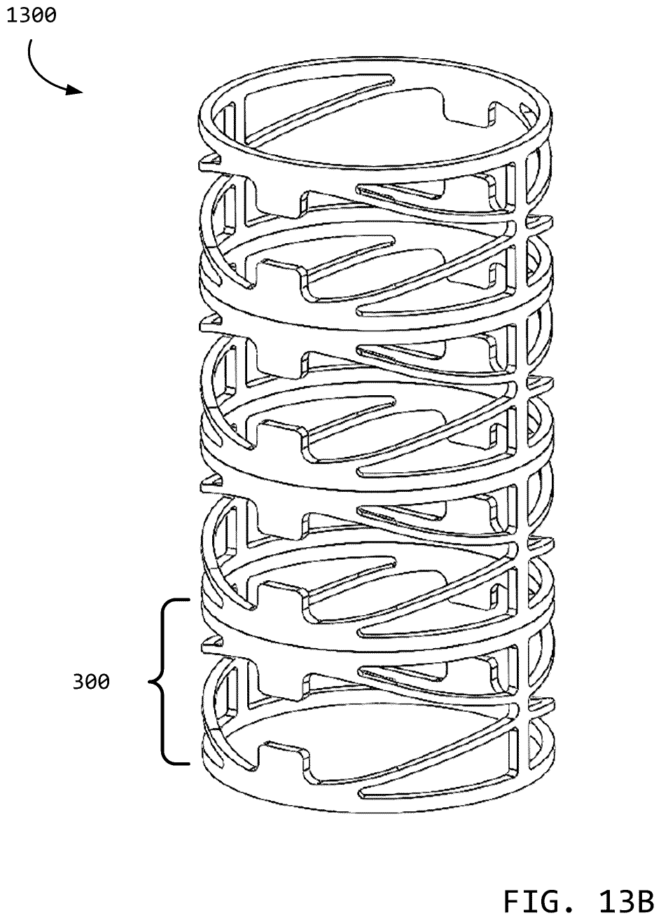

FIGS. 13A-13B illustrate flexible frameworks 1300, composed of flexible framework components 300 (e.g., two or more of any flexible framework component 300 as shown in FIGS. 3-11 and the variations thereof discussed in relation to the associated Figures). The illustrated embodiment includes seven flexible framework components 300, but a flexible framework 1300 may include any number of flexible framework components 300, in order to meet the demands of a desired usage. Flexible frameworks 1300 may include about five, ten, twenty, fifty, one hundred, or five hundred flexible framework components 300, in various embodiments, although more or fewer may be included.

The individual framework components 300 may be any one of the flexible framework components 300 shown in FIGS. 3-11. Flexible Frameworks 1300 may also include embodiments of flexible framework components having some or all of the discussed variants of dependent bend limiters 420, independent bend limiters 520, wave-matched bend limiters 610, and protrusions 710 with guide holes 725. Furthermore, a single flexible framework 1300 may also include multiple embodiments of flexible framework components, such as those listed above. Stated differently, any successive flexible framework component 300 in a flexible framework 1300 may have any quantity of guide holes 715, a different points of intersection of two components, or a height that is different from a respective quantity of through holes, a point of intersection of two components, or a height than a neighboring flexible framework component 300. In various embodiments, the framework 1300 can be 3-D printed or laser cut from tubing, and can be made out of polymer, metal, etc.

In some embodiments the flexible framework components 300 in a flexible framework 1300 are connected by means of fasteners, which may include bolts, screws, rivets, pins and the like. In some embodiments, the flexible framework components 300 in a flexible framework 1300 are connected by heat bonding, welding, brazing or one of various adhesives. In some embodiments, the flexible framework components 300 in a flexible framework 1300 are connected by integrated manufacture.

In some examples, a flexible framework 1300 includes a protective coating that coats each individual flexible framework component of the flexible framework. This coating may be a protective sealant, or an applied layer of finish.

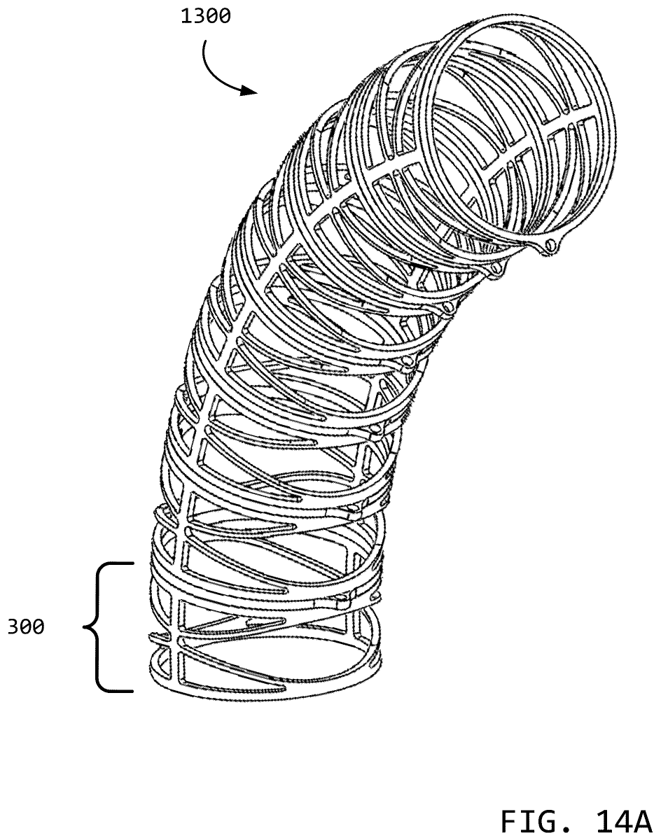

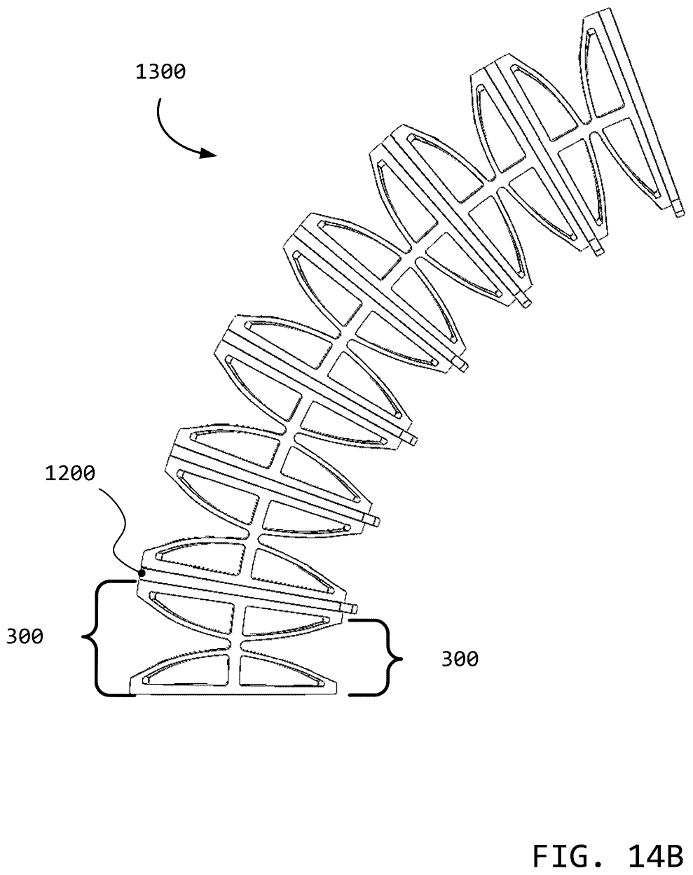

FIG. 14A illustrates a flexible framework 1300 in bending. The axial members 330 of each flexible framework component 300 collectively define a neutral axis for the entire flexible framework 1300. In the illustrated embodiment, the axial members 330 of each flexible framework component 300 are axially aligned, and the flexible framework 1300 has a uniform axis of bending in the Z-direction. The axial members 330 of each individual flexible framework component 300 ensure that the axial arc length of the deflected flexible framework 1300 is equal to that of the height of the non-deflected flexible framework 1300.

FIG. 14B illustrates a planar X-Y view of a flexible framework 1300 in bending.

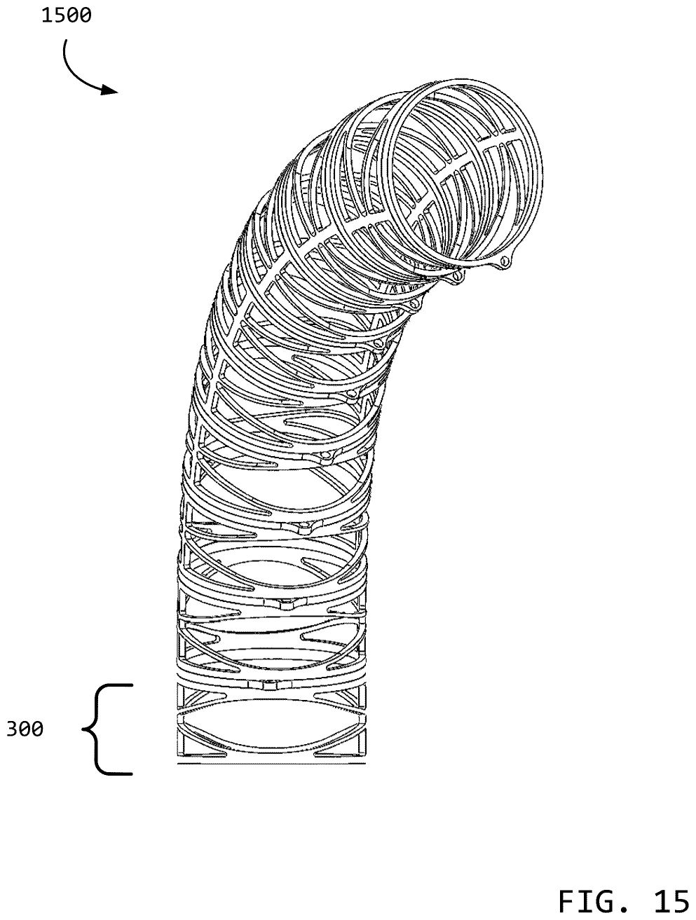

FIG. 15 shows a planar Y-Z view of a flexible framework 1300 where each flexible framework component 300 is rotationally offset from one another. By rotationally offsetting each flexible framework component 300, the axial curve of the flexible framework 1300 in bending is no longer two-dimensional. When the flexible framework components 300 are no longer rotationally aligned, the neutral axes of each flexible framework component 300 are no longer axially aligned. This results in each flexible framework component 300 deflecting in a different direction relative to a first flexible framework component, and irregular curves and unique formations of the flexible framework 1300 can be created.

FIG. 16 illustrates a wire guided flexible framework 1600. In some embodiments, a wire guided flexible framework 1500 includes flexible framework components 300 with guide holes 720 and a wire 1610 disposed within the guide holes 720. The wire 1610 is installed through each axially aligned guide hole 720 in a wire guided flexible framework 1600, parallel to the axial members 330. The wire 1610 is secured to the final flexible framework component and passes through the guide holes 720 of the other flexible framework components. The unattached end of the wire 1610 may be attached to an actuator, or operated manually. When tension is introduced to the wire 1610 by retraction, the attached end of the wire 1610 transfers a compressive force to the final flexible framework component. As the tension is increased and the wire 1610 is shortened, the flexible components of the wire guided flexible framework 1600 deflect towards the side of the framework in which the wire 1610 is disposed. In embodiments where the neutral axes of each flexible framework component are aligned, the wire guided flexible framework 1600 deflects as illustrated in FIGS. 14A-14B. In alternate terms, shortening the wire 1610 provides the compressive force required for the wire guided flexible framework 1600 to deflect.

According to some embodiments, wire guided flexible frameworks 1600 are configured to have a tube disposed within the framework, for the purposes of fluid transport from one end of the framework to the other. In some such embodiments, the tube is disposed in the empty space inside each flexible framework component (See FIG. 17B). In other such embodiments, the tube is disposed within the central holes 720 of disks 700 disposed between each successive flexible framework component 300.

According to some embodiments, wire guided flexible frameworks 1500 do not include flexible framework components 300 with guide holes 715. Alternatively, the wire 1610 is disposed in circumferential holes 1210 of disks, wherein a disk is disposed in between each flexible component of the wire guided flexible framework 1500.

FIG. 17A shows a wire guided flexible framework 1600 disposed within a flexible tube 1710 (e.g., a jacket). According to some embodiments, the tube is a catheter. In such embodiments, the flexible framework may be a wire guided flexible framework 1600, such that the catheter is operable to be guided during use. In some examples, the tube is a mesh tube, a textile tube, or a polymer tube disposed about an outer circumference of the flexible framework. In some embodiments the tube can be a jacket that is installed over the tube, where the jacket is a section of mesh or textile that is secured about the framework.

FIG. 17B illustrates a wire guided flexible framework 1600 disposed within an external flexible tube 1710, and having an internal flexible tube 1720 (e.g., a liner) disposed within the empty space inside the wire guided flexible framework 1600. The internal tube may be used for fluid transport, such as in a cathing process. In the illustrated embodiments, the wire guided flexible framework 1600 includes external protrusions 710 and guide holes 720 through which the wire 1610 passes. In some embodiments, the wire guided flexible framework 1600 may include wire guide holes 720 not aligned with the bending plane of the individual flexible framework components 300. Together, the embodiments shown in FIGS. 17A and 17B form a catheter, although the two maybe be used separately in different devices.

For effective multi-directional deflection with rotationally offset flexible framework components 300, some wires 1610 must traverse sections of the wire guided flexible framework 1600 in which another wire 1610 is responsible for the bending. According to some embodiments, additional protrusions 710 and guide holes 720 on given flexible framework component 300 may serve as retaining points for a wire 1610 that does not induce deflection in the given flexible framework component 300, but induces deflection in other flexible framework components 300 in the wire guided flexible framework 1600. These additional protrusions 710 and guide holes 720, may be located at any point about an annular member 310 of a given flexible framework component 300, in order to serve the need of the wire guided flexible framework 1600.

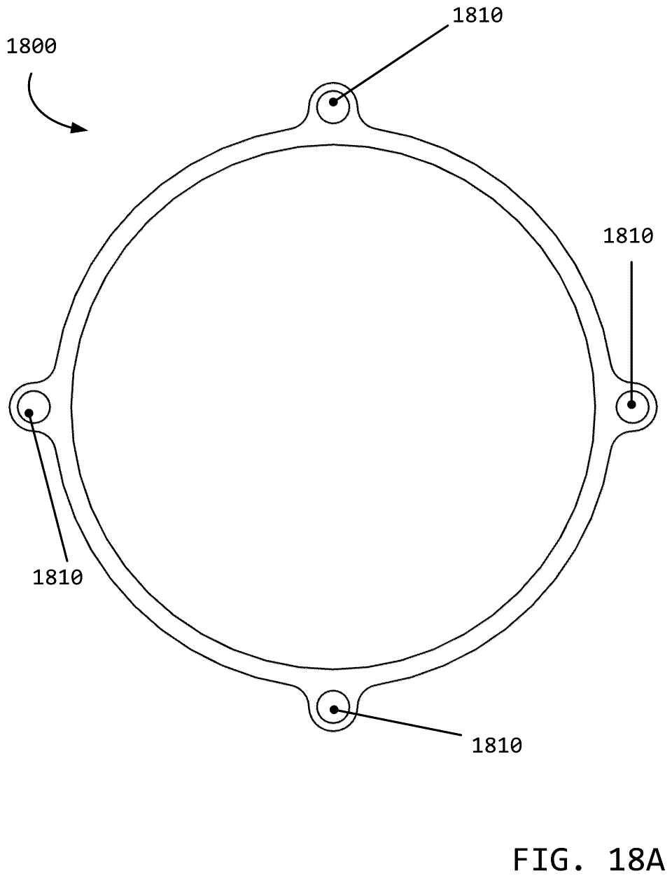

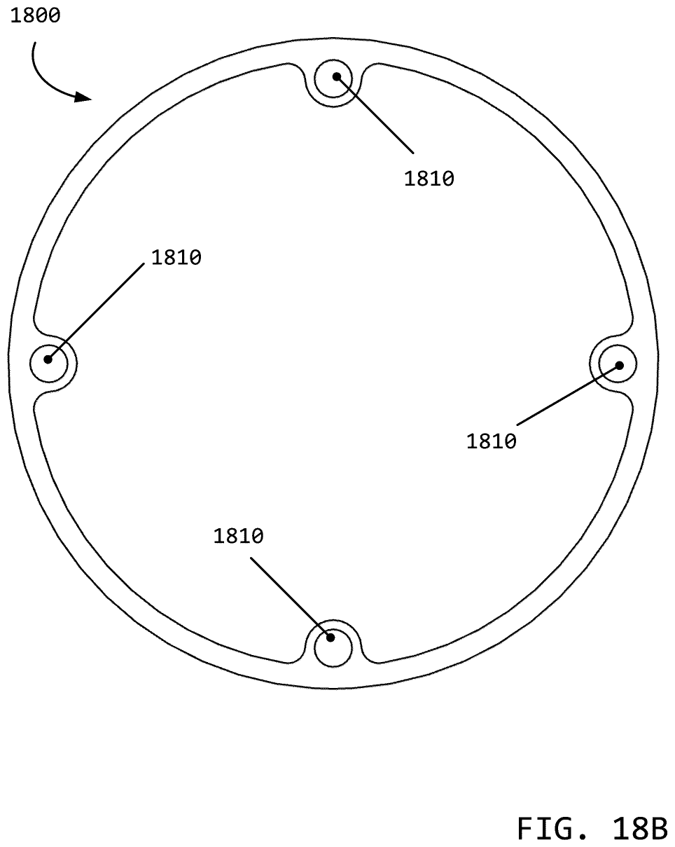

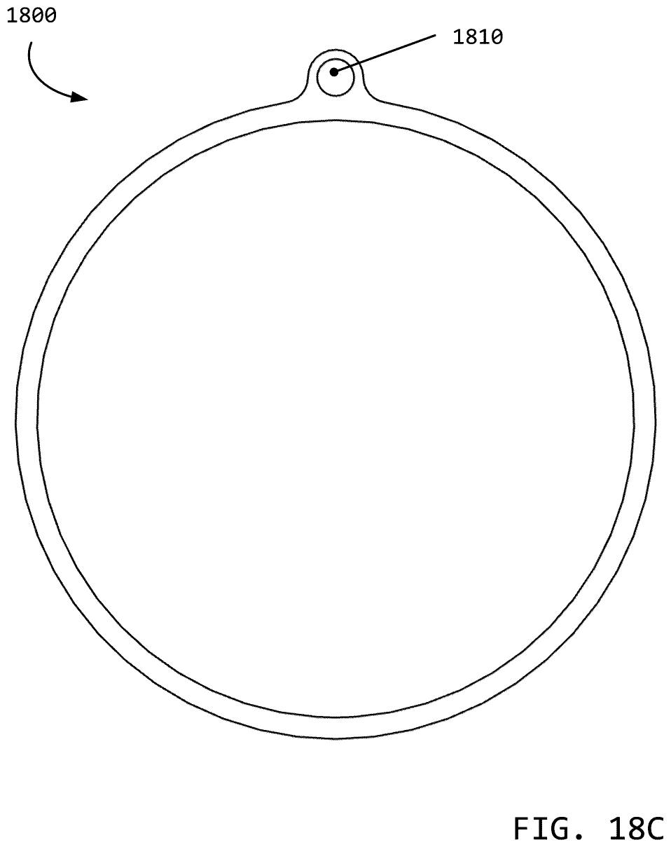

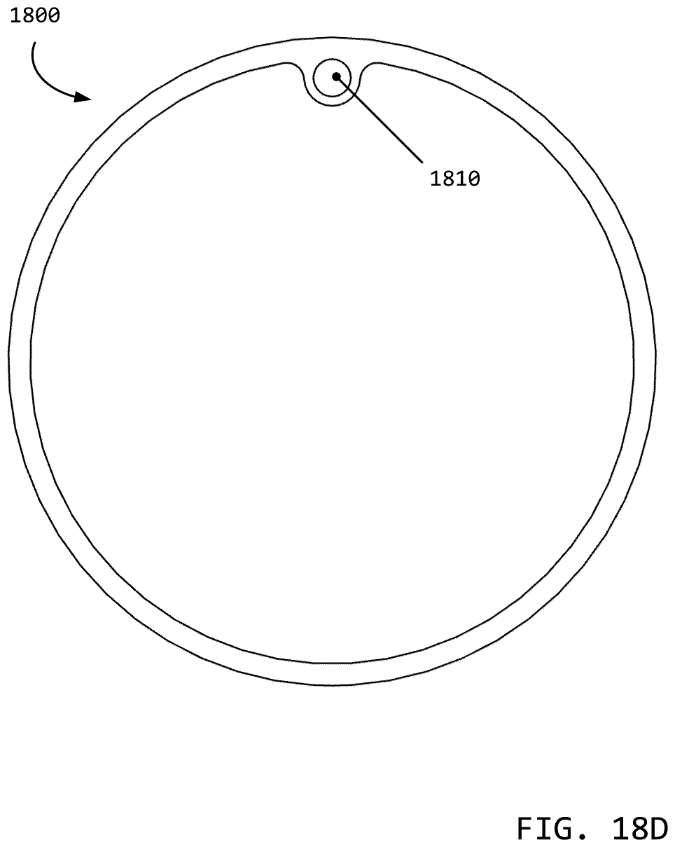

FIG. 18A-18D illustrate example wire guide elements 1800 with wires guides 1810 in various locations, according to embodiments of the present disclosure. The present disclosure contemplates that various wire guide elements 1800 may be used as integrated elements of the flexible framework components 300 or as separate elements secured to one or more flexible framework components 300. For example, FIGS. 7A and 7B show internal and external wire guides as a single piece, whereas FIGS. 14A through 16 show wire guides that are individual pieces that are disposed between the individual framework components 300. FIGS. 18A-18B show that a variety of number and positions of wire guides 1810 may be included in a wire guide element 1800, and that different numbers and orientations of wire guides 1810 can be included at different locations within a single wire guided flexible framework 1600 or on opposing ends (integrated or separately attached) of a single flexible framework component 300.

Certain terms are used throughout the description and claims to refer to particular features or components. As one skilled in the art will appreciate, different persons may refer to the same feature or component by different names. This document does not intend to distinguish between components or features that differ in name but not function.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, A-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112 (f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims

The invention is claimed as follows:

1. A flexible framework, comprising a plurality of flexible components, each flexible component of the plurality of flexible components having a plurality of structural members, including:
a first annular member;
a second annular member;
a first axial member, connected on opposing ends to the first annular member and the second annular member, respectively;
a second axial member, connected on opposing ends to the first annular member and the second annular member, respectively;
a first undulating member, having an annular profile and a first waveform defined about a circumference thereof, which connects to the first annular member twice, connects to the first axial member once, and connects to the second axial member once; and
a second undulating member, having an annular profile and a second waveform defined about a circumference thereof, which connects to the second annular member twice, connects to the first axial member once, and connects to the second axial member once.

2. The flexible framework of claim 1, wherein for a given flexible component of the plurality of flexible components:
the first undulating member and the second undulating member intersect the first axial member at a shared point on the first axial member; and
the first undulating member and the second undulating member intersect the second axial member at a shared point on the second axial member.

3. The flexible framework of claim 1, wherein for a given flexible component of the plurality of flexible components:
the first undulating member and the second undulating member intersect the first axial member at respectively distinct points on the first axial member; and
the first undulating member and the second undulating member intersect the second axial member at respectively distinct points on the second axial member.

4. The flexible framework of claim 1, wherein for a given flexible component of the plurality of flexible components:
the given flexible component further comprises a protrusion on an inner circumference of the first annular member, wherein the protrusion defines a through hole.

5. The flexible framework of claim 1, further comprising a plurality of disks having a plurality of through holes defined therein, wherein each disk is disposed between two flexible components.

6. The flexible framework of claim 1, wherein for a given flexible component of the plurality of flexible components, the given flexible component further comprises a bend limiter, disposed within a plane of deflection, configured to limit an extent of deflection of the given flexible component.

7. The flexible framework of claim 6, wherein the bend limiter comprises one of:
a first dependent bend limiter projecting from the first annular member and paired with a second dependent bend limiter projecting from the second annular member;
an independent bend limiter projecting from the first annular member and not paired with a corresponding bend limiters projection from the second annular member;
a wave-matched bend limiter defined in a third annular member, disposed between the first annular member and the second annular member, and having a shape matched to the first waveform and the second waveform; and
a positive protrusion projecting from the first annular member and paired with a negative protrusion defined within the second annular member.

8. The flexible framework of claim 1, wherein for a given flexible component of the plurality of flexible components, the given flexible component is formed by removing material from a section of pipe or tubing.

9. The flexible framework of claim 1, wherein for a given flexible component of the plurality of flexible components, the given flexible component is formed by additive manufacturing.

10. The flexible framework of claim 1, wherein a first flexible component is connected to a second flexible component via one or more of welding, brazing, use of an adhesive, integrated manufacture, and fasteners.

11. The flexible framework of claim 1, wherein at least one of a height, a quantity of through holes, or a point of intersection of two structural components of a first flexible component is different from a corresponding one of a height, a quantity of through holes, or a point of intersection of two structural components of a second flexible component.

12. The flexible framework of claim 1, wherein a neutral axis of a first flexible component is rotationally offset from a neutral axis of a second flexible component, such that when the flexible framework is subjected to a bending force, the flexible framework deflects into an irregular curve.

13. The flexible framework of claim 1, wherein the flexible framework includes a coating, comprising one of a sealant, a finish, and a protective layer that coats each flexible component of the plurality of flexible components.

14. The flexible framework of claim 1, wherein the flexible framework includes a jacket, comprising one of a polymer tube, a textile tube, and a mesh tube disposed around an outer circumference of the flexible framework.

15. The flexible framework of claim 1, wherein a tube is disposed within the flexible framework in a space defined within respective first annular members and second annular members of each flexible component of the plurality of flexible components.

16. The flexible framework of claim 1, further comprising a wire, configured such that the wire passes through a through hole in each of the flexible components, and when a tension is induced in the wire, bending is induced in the flexible framework.

17. The flexible framework of claim 1, wherein the first axial member and the second axial member each comprise compliant structures that comprise undulations that define one or more gaps of material along straight-line paths defined between the first annular member and the second annular member.

18. The flexible framework of claim 1, wherein for a given flexible component of the plurality of flexible components:
a third annular member is disposed between the first annular member and the second annular member, wherein a first height between the first annular member and the third annular member is different from a second height between the second annular member and the third annular member.

19. The flexible framework of claim 1, wherein for a given flexible component of the plurality of flexible components:
a third annular member is disposed between the first annular member and the second annular member, wherein inter-annular heights between the first annular member and the third annular member and between the second annular member and the third annular member are substantially equivalent;
the first annular member defines a first positive protrusion;
the third annular member defines a first negative protrusion disposed corresponding to the first positive protrusion and defines a second positive protrusion;
the second annular member defines a second negative protrusion disposed corresponding to the second positive protrusion, wherein a first height defined between the first positive protrusion and the first negative protrusion is different from a second height defined between the second positive protrusion and the second negative protrusion.

20. The flexible framework of claim 1, wherein for a given flexible component of the plurality of flexible components:
wherein the first undulating member is connected to the first annular member via a first axial offset and a second axial offset, wherein the first axial offset and the second axial offset extend from the first annular member towards the second annular member.

21. The flexible framework of claim 1, wherein for a given flexible component of the plurality of flexible components:
the second axial member is connected to the first annular member and the second annular member diametrically opposite to where the first axial member is connected to the first annular member and the second annular member.

22. The flexible framework of claim 1, wherein for a given flexible component of the plurality of flexible components:
the second axial member is connected to the first annular member and the second annular member unevenly about circumferences to where the first axial member is connected to the first annular member and the second annular member, wherein the given flexible component exhibits a greater degree of flexible in a first direction in which the first axial member is located further from the second axial member along the circumference than in a second direction in which the first axial member is closer further from the second axial member along the circumferences.

23. A flexible component, comprising:
a first annular member;
a second annular member;
a first axial member, connected on opposing ends to the first annular member and the second annular member, respectively;
a second axial member, connected on opposing ends to the first annular member and the second annular member, respectively, diametrically opposite from where the first axial member is connected to the first annular member and the second annular member;
a first undulating member, having an annular profile and a waveform defined about a circumference thereof, which intersects the first annular member twice, intersects the first axial member once, and intersects the second axial member once; and
a second undulating member, having an annular profile and a waveform defined about a circumference thereof, which intersects the second annular member twice, intersects the first axial member once, and intersects the second axial member once.

24. The flexible component of claim 23, further comprising:
a plurality protrusions on an inner circumference of the first annular member or the second annular member, each protrusion having a though-hole disposed therein.

25. The flexible component of claim 23, wherein the first axial member and the second axial member control a direction of deflection by forming neutral axes when the flexible component is subject to a bending stress.

26. The flexible component of claim 23, wherein the first axial member and the second axial member each comprise compliant structures that comprise undulations that define one or more gaps of material along straight-line paths defined between the first annular member and the second annular member.

27. The flexible component of claim 23, further comprising a bend limiter that includes one of:
a first dependent bend limiter projecting from the first annular member and paired with a second dependent bend limiter projecting from the second annular member;
an independent bend limiter projecting from the first annular member and not paired with a corresponding bend limiters projection from the second annular member;
a wave-matched bend limiter defined in a third annular member, disposed between the first annular member and the second annular member, and having a shape matched to the first waveform and the second waveform; and
a positive protrusion projecting from the first annular member and paired with a negative protrusion defined within the second annular member.

28. The flexible component of claim 23, further comprising a third annular member disposed between the first annular member and the second annular member, wherein inter-annular heights between the first annular member and the third annular member and between the second annular member and the third annular member are substantially equivalent;
the first annular member defines a first positive protrusion;
the third annular member defines a first negative protrusion disposed corresponding to the first positive protrusion and defines a second positive protrusion;
the second annular member defines a second negative protrusion disposed corresponding to the second positive protrusion, wherein a first height defined between the first positive protrusion and the first negative protrusion is different from a second height defined between the second positive protrusion and the second negative protrusion.

29. A wire-guidable flexible framework comprising a plurality of flexible framework components and a plurality of wires, wherein:
each flexible framework component comprises a neutral axis component, a waveform component, and an annular structural component having a protrusion containing a though hole; and
the plurality of flexible components are secured together into the flexible framework, and configured such that a wire of the plurality of wires passes through a through hole in each of the flexible components, and when a tension is induced in the wire, bending is induced in the flexible framework.

30. The wire-guidable flexible framework of claim 29, wherein a given protrusion on a given flexible framework component is disposed 90 degrees about a given annular structural component from an intersection of the neutral axis component on the given annular structural component.

31. The wire-guidable flexible framework of claim 29, wherein a neutral axis component comprises compliant structures that comprise undulations that define one or more gaps of material along straight-line paths defined between neighboring annular structural components.

32. The wire-guidable flexible framework of claim 29, the annular structural component further comprising a bend limiter that includes one of:
a first dependent bend limiter projecting from the annular structural component and paired with a second dependent bend limiter projecting from a second annular structural component;
an independent bend limiter projecting from the annular structural component and not paired with a corresponding bend limiters projecting from the second annular structural component;
a wave-matched bend limiter defined in a third annular structural component, disposed between the annular structural component and the second annular structural component, and having a shape matched to a shape of the waveform component; and
a positive protrusion projecting from the annular structural component and paired with a negative protrusion defined within the second annular structural component.

33. The wire-guidable flexible framework of claim 29, further comprising a third annular structural component disposed between the annular structural component and a second annular structural component, wherein inter-annular heights between the annular structural component and the third annular structural component and between the second annular structural component and the third annular structural component are substantially equivalent;
the annular structural component defines a first positive protrusion;
the third annular structural component defines a first negative protrusion disposed corresponding to the first positive protrusion and defines a second positive protrusion;
the second annular structural component defines a second negative protrusion disposed corresponding to the second positive protrusion, wherein a first height defined between the first positive protrusion and the first negative protrusion is different from a second height defined between the second positive protrusion and the second negative protrusion.

34. The wire-guidable flexible framework of claim 29, wherein a neutral axis component comprises an axial member connected on a first end to the annular structural component and on a second end to the waveform component, wherein a neutral axis gap is defined between the waveform component and a second annular member of the flexible framework component.
